(19) United States
(12) Patent Application Publication
(10) Pub. No.: US 2019/0276414 A1
(43) Pub. Date: Sep. 12, 2019

(54) CHIRAL RESOLUTION OF AN INTERMEDIATE OF SUVOREXANT AND COCRYSTALS THEREOF

(71) Applicant: ENANTIA, S.L., BARCELONA (ES)

(72) Inventors: Alexander Christian COMELY, BARCELONA (ES); Nicolas TESSON, L'HOSPITALET DE LLOBREGAT (ES)

(21) Appl. No.: 16/304,634

(22) PCT Filed: Jun. 2, 2017
(86) PCT No.: PCT/EP2017/063515

§ 371 Date: Nov. 26, 2018

(30) Foreign Application Priority Data
Jun. 6, 2016 (EP) 16382260.4

(51) Int. Cl. C07D 243/08; C07C 39/16; C07D 413/14; C07B 57/00

(52) U.S. Cl. CPC C07D 243/08; C07C 39/16; C07B 2200/07; C07B 57/00; C07B 2200/13; C07D 413/14

(57) ABSTRACT

Relating to processes for preparing suvorexant or its pharmaceutically acceptable salts through the formation of a cocrystal of (R)-benzyl 5-methyl-1,4-diazepane-1-carboxylate hydrochloride with (R)-(+)-1,1,2-triphenyl-1,2-ethanediol ((R)-TED). This cocrystal provides the resolution of an intermediate of suvorexant, in particular, of (rac)-benzyl5-methyl-1,4-diazepane-1-carboxy-lateor a hydrochloride salt thereof. It also relates to a new cocrystal useful in such preparation processes.

This application claims the benefit of European Patent Application EP16382260.4 filed 6 Jun. 2016.

### TECHNICAL FIELD

The present invention relates to processes for preparing suvorexant or its salts through the resolution of (rac)-benzyl 5-methyl-1,4-diazepane-1-carboxylate which is an intermediate of suvorexant. It also relates to new cocrystals useful in such preparation processes.

### BACKGROUND ART

Suvorexant (MK-4305) is the International Non-proprietary Name (INN) of [(7R)-4-(5-chloro-1,3-benzoxazol-2-yl)-7-methyl-1,4-diazepan-1-yl][5-methyl-2-(2H-1,2,3-triazol-2-yl)phenyl]methanone and its CAS number is 1030377-33-3. It is currently marketed as Belsomra and is a selective, dual orexin receptor antagonist for the treatment of insomnia.

The structure of suvorexant corresponds to formula (I) below.

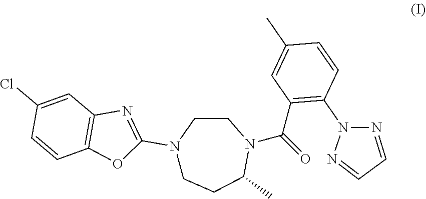 [SMILES: Cc1ccc(-n2nccn2)c(C(=O)N2CCN(c3nc4cc(Cl)ccc4o3)CC[C@H]2C)c1]

Suvorexant has one chiral center with a configuration (R). The synthesis of suvorexant is described in the patent family of WO2008069997A1 and in Cox et al., J. Med. Chem. 2010, vol. 53, pp. 5320-5332. In particular, a synthesis that is based on a chiral stationary phase HPLC resolution of a racemic 1,4-diazepane derivative ((rac)-(VIIIa)) is described (see scheme 1).

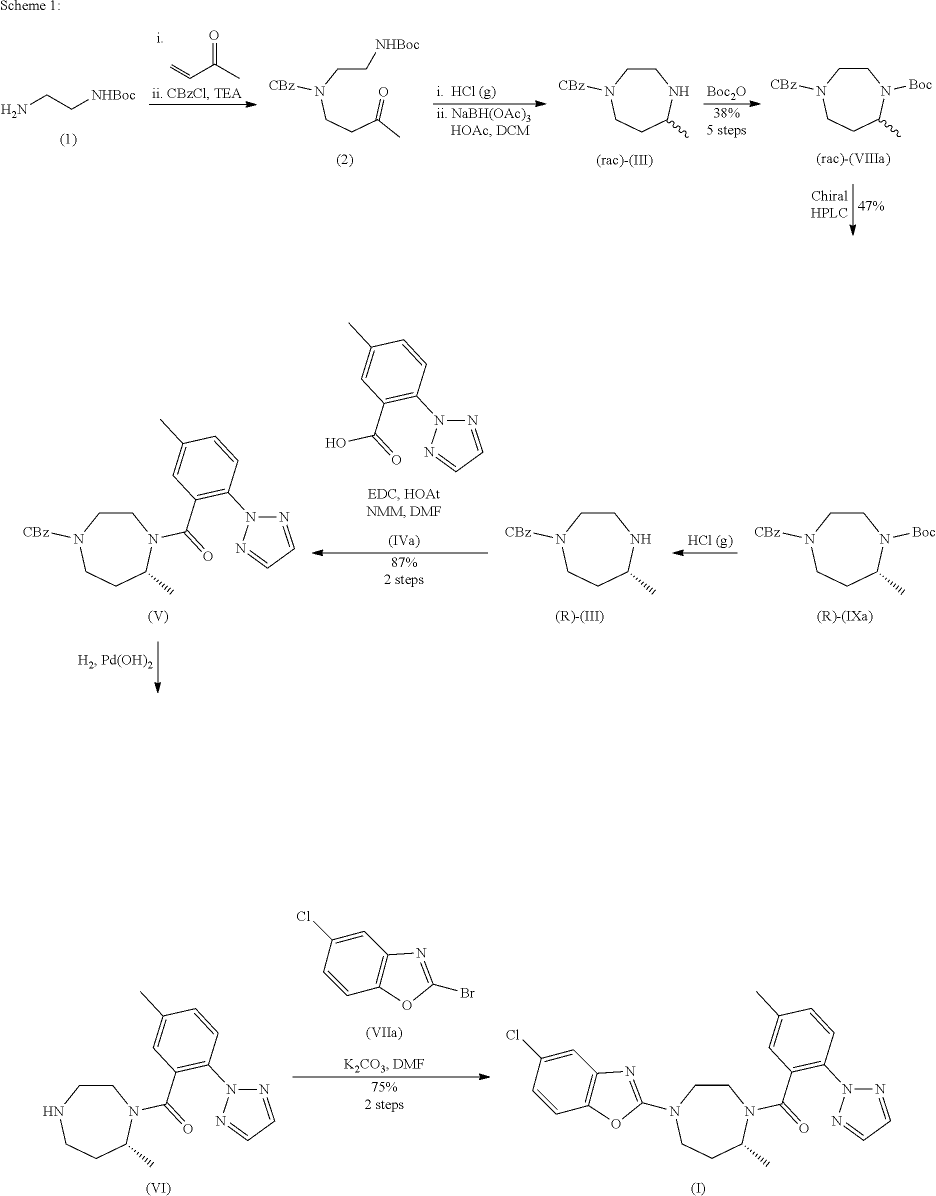

However, the chiral resolution of enantiomers of the 1,4-diazepane derivative ((rac)-(VIIIa)) via chiral HPLC is not adapted to an industrial process and has several drawbacks: moderate throughput, large amount of solvent, high cost, and large amount of waste.

In WO2016020404A1, the resolution of different possible intermediates of suvorexant is disclosed based on the formation of diastereomeric salts with tartaric acid derivatives such as 2,3-ditoluoyl tartaric acid, 2,3-dibenzoyl tartaric acid, 2,3-dianisoyl tartaric acid, and 2,3-dibenzoyl tartaric acid mono(dimethylamide). Amongst these possible intermediates, resolution of (rac)-(III) was performed by formation of a diastereomeric salt with 2,3-dibenzoyl D-tartaric acid (DBTA) (see Scheme 2).

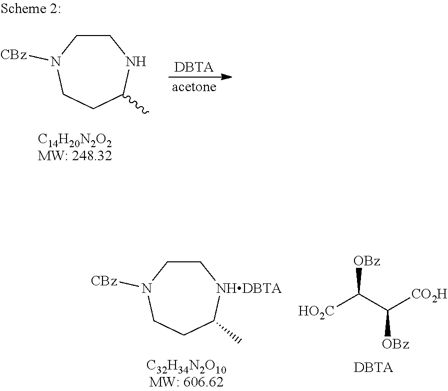

Two examples of resolution of (rac)-benzyl 5-methyl-1,4-diazepane-1-carboxylate with DBTA were described (see page 69-70 of WO2016020404A1). One on a small scale (1.25 g) where crystallization of (rac)-benzyl 5-methyl-1,4-diazepane-1-carboxylate with DBTA (1 eq.) in acetone afforded benzyl 5-methyl-1,4-diazepane-1-carboxylate.DBTA salt with 18% yield and 93.4% ee (enantiomeric excess). Another at larger scale (7.26 g) where crystallization of (rac)-benzyl 5-methyl-1,4-diazepane-1-carboxylate with DBTA (0.5 eq.) in acetone afforded benzyl 5-methyl-1,4-diazepane-1-carboxylate.DBTA salt with a higher yield of 31% but a lower 76.6% ee. The recrystallization of this last compound in ethanol afforded the compound with 64% yield and 95.4% ee. The global yield to obtain the compound with 95.4% ee is 19.8%. Thus, in both examples, (R)-benzyl 5-methyl-1,4-diazepane-1-carboxylate.DBTA was obtained with a low yield (18-19%) and with <96% ee. As the enantiomeric excess of these examples remains low, one or more additional recrystallizations should be performed to reach 98-99% ee, decreasing even further the low yield of this resolution.

Hence, there is the need to provide an efficient method for the resolution of (rac)-benzyl 5-methyl-1,4-diazepane-1-carboxylate, which is an intermediate of suvorexant, with improved yield and higher % ee.

### SUMMARY OF THE INVENTION

The inventors have developed a process for the resolution of (rac)-benzyl 5-methyl-1,4-diazepane-1-carboxylate ((rac)-(III)), or a salt thereof, such as the hydrochloride salt, by formation of a cocrystal of (R)-benzyl 5-methyl-1,4-diazepane-1-carboxylate hydrochloride with (R)-(+)-1,1,2-triphenyl-1,2-ethanediol ((R)-TED).

According to the inventors' knowledge, the use of cocrystals for the resolution of suvorexant intermediates has not been disclosed in the prior art. The identification of a cocrystal for use as a resolution agent is not a routine exercise and the formation of a cocrystal cannot be predicted theoretically. Other chiral 1,2-diols were also tested by the inventors without success. Thus, the formation of a cocrystal of (R)-benzyl 5-methyl-1,4-diazepane-1-carboxylate hydrochloride with (R)-TED, of formula (II) is considered a contribution to the art.

Accordingly, an aspect of the present invention relates to a cocrystal of (R)-benzyl 5-methyl-1,4-diazepane-1-carboxylate hydrochloride with (R)-TED, of formula (II) wherein Cbz is benzyloxycarbonyl, which has a molar ratio of (R)-benzyl 5-methyl-1,4-diazepane-1-carboxylate hydrochloride: (R)-TED of 1:1.

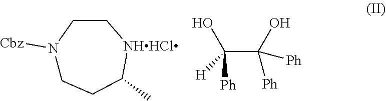

Another aspect of the present invention relates to a resolution process comprising the preparation of a cocrystal of (R)-benzyl 5-methyl-1,4-diazepane-1-carboxylate hydrochloride ((R)-(III).HCl) with (R)-TED, of formula (II) wherein Cbz is benzyloxycarbonyl, by a process which comprises: a₁) combining a₁ₐ) either a mixture of a hydrochloride salt of (rac)-benzyl 5-methyl-1,4-diazepane-1-carboxylate of formula (rac)-(III) and (R)-TED or, alternatively, a_{1b}) (rac)-benzyl 5-methyl-1,4-diazepane-1-carboxylate of formula (rac)-(III), (R)-TED, and hydrochloric acid; in a solvent selected from the group consisting of acetonitrile, isopropanol, ethyl acetate, acetone, tetrahydrofuran, tert-butyl methyl ether, and toluene; a₂) either heating the mixture until complete dissolution or, alternatively, slurrying between room temperature and reflux; a₃) cooling down this mixture, if necessary; and a₄) isolating the cocrystal of steps a₂) or a₃) thus obtained.

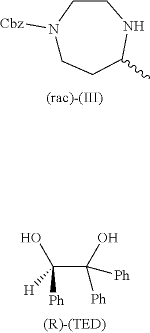 [SMILES: CC1CCN(C(=O)OCc2ccccc2)CCN1 ; O[C@H](c1ccccc1)C(O)(c1ccccc1)c1ccccc1]

The cocrystal obtained in step a₂) or a₃) is the cocrystal of (R)-benzyl 5-methyl-1,4-diazepane-1-carboxylate hydrochloride with (R)-TED of formula (II).

In a particular embodiment, of the previous process, the salt is the hydrochloride salt.

The previous process allows performing the resolution of (rac)-benzyl 5-methyl-1,4-diazepane-1-carboxylate of formula (rac)-(III) or a salt thereof. The process may comprise converting the cocrystal of step a₄) into (R)-benzyl 5-methyl-1,4-diazepane-1-carboxylate or a salt thereof.

The resolution process of (rac)-benzyl 5-methyl-1,4-diazepane-1-carboxylate or a salt thereof, in particular of a hydrochloride salt thereof, may comprise: a) preparing a cocrystal of (R)-benzyl 5-methyl-1,4-diazepane-1-carboxylate hydrochloride with (R)-TED of formula (II) by the process disclosed above, optionally purifying the cocrystal thus obtained by either recrystallization or by slurrying in an organic solvent, and b) dissociating the cocrystal thus obtained to yield (R)-benzyl 5-methyl-1,4-diazepane-1-carboxylate hydrochloride salt, if desired converting it to its free base of formula (R)-(III); and, if desired, c) converting the resulting free base into a salt thereof by reacting it with an acceptable acid.

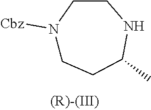 [SMILES: C[C@@H]1CCN(C(=O)OCc2ccccc2)CCN1]

The previous process when comprising one recrystallization affords very high % ee (>99% ee with only one recrystallization) with a satisfactory yield around 25-30%. Furthermore, the same solvent can be used for two of the steps of the resolution process (formation of the cocrystal for instance by crystallization, and then recrystallization). An additional advantage is the easy recovery of TED during the dissociation step. The resolution process may be carried out from (rac)-benzyl 5-methyl-1,4-diazepane-1-carboxylate or a hydrochloride salt thereof or from a mixture of (R) and (S) enantiomers of any of these compounds in other ratios.

Another aspect of the present invention relates to the use of the cocrystal of (R)-benzyl 5-methyl-1,4-diazepane-1-carboxylate hydrochloride with (R)-TED, of formula (II) as defined above, as an intermediate for the preparation of (R)-suvorexant or a pharmaceutically acceptable salt thereof.

Also part of the invention are processes for preparing suvorexant of formula (I), or a pharmaceutically acceptable salt thereof, which comprise a) carrying out the resolution process as disclosed above, and converting the compound thus obtained into suvorexant or its pharmaceutically acceptable salts by methods disclosed in the state of the art which are disclosed in detail below. These processes have better industrial applicability and utility than the known processes for preparing suvorexant and overcome the drawbacks of the existing ones.

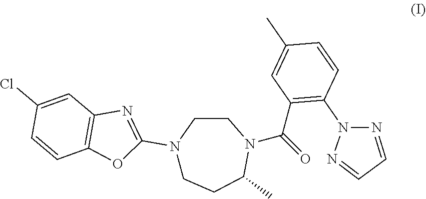 [SMILES: Cc1ccc(-n2nccn2)c(C(=O)N2CCN(c3nc4cc(Cl)ccc4o3)CC[C@H]2C)c1]

### BRIEF DESCRIPTION OF THE DRAWINGS

FIG. 1 shows the XRPD of the cocrystal of (R)-benzyl 5-methyl-1,4-diazepane-1-carboxylate.HCl with (R)-TED named Form A.

FIG. 2 shows the ¹H NMR of Form A.

FIG. 3 shows the DSC of Form A.

FIG. 4 shows the TGA of Form A

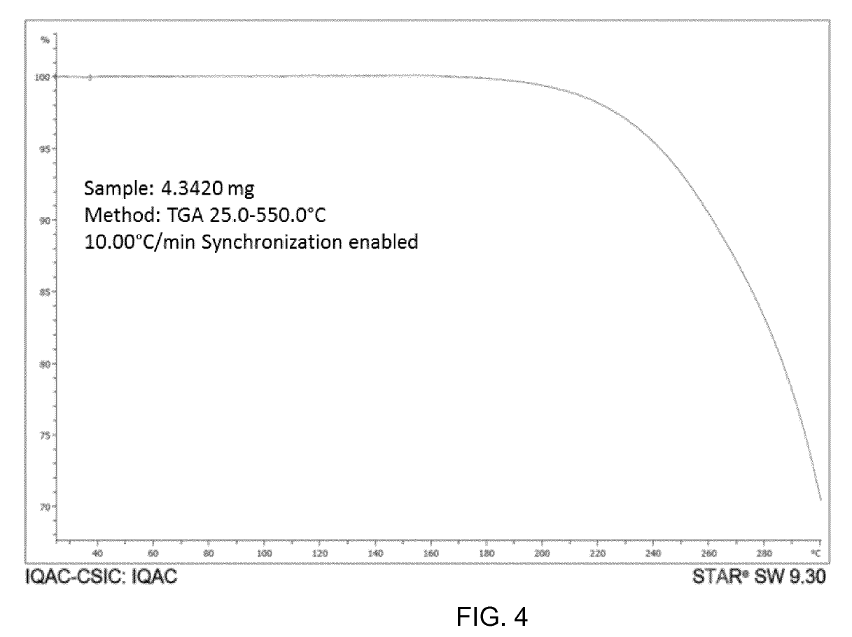

### DETAILED DESCRIPTION OF THE INVENTION

For the sake of understanding, the following definitions are included and expected to be applied throughout the description, claims and drawings.

Unless stated otherwise, the term “suvorexant” in the context of the present invention relates to (R)-suvorexant. Both terms have been used interchangeably.

The term “cocrystal” refers herein to a crystalline entity with at least two different components constituting the unit cell at room temperature (20-25° C.) and interacting by weak interactions. Thus, in a cocrystal the target molecule crystallizes with one or more neutral components. The cocrystals may include one or more solvent molecules in the crystal lattice.

The term “weak interaction” refers herein as an interaction which is neither ionic nor covalent, and includes for example: hydrogen bonds, van der Waals interactions, and π-π stacking.

When a ratio of components of the cocrystals of the invention is specified it refers to the molar ratio between the two components that form the cocrystal. The term “molar ratio” has been used to express the stoichiometric amount in moles of each of the components of a cocrystal.

When values of characteristic peaks of an X-ray diffractogram are given it is said that are “approximate” values. It should be understood that the values are the ones shown in the corresponding lists or tables ±0.3 degrees 2 theta measured in an X-ray diffractometer with Cu—K_{α} radiation λ=1.5406 Å.

The term “room temperature” as disclosed herein refers to a temperature of the environment, without heating or cooling, and is generally comprised of from 20 to 25° C.

For the purposes of the invention, any ranges given include both the lower and the upper end-points of the range. Ranges given, such as temperatures, times and the like, should be considered approximate, unless specifically stated.

Enantiomeric excess (ee) is a measurement of purity used for chiral substances. It reflects the degree to which a sample contains one enantiomer in greater amounts than the other. The term “enantiomeric purity” (or optical purity) is defined as the fractional excess of one enantiomer over the other.

The expression “cocrystal obtainable by” is used here to define each specific cocrystal of the invention by the process for obtaining it and refers to the product obtainable by any of the corresponding processes disclosed herein. For the purposes of the invention the expressions “obtainable”, “obtained” and equivalent expressions are used interchangeably and, in any case, the expression “obtainable” encompasses the expression “obtained”.

The terms “wet grinding” and “liquid assisted grinding” are equivalent and refer to a technique which consists of milling or grinding the product or mixture with some drops of solvent added. Neat and liquid-assisted grinding are techniques that can be employed in order to produce cocrystals. In neat (dry) grinding, cocrystal formers are ground together manually using a pestle and mortar, using a ball mill, or using an oscillatory mill. In liquid-assisted grinding, or kneading, a small amount of liquid (solvent), for instance, some drops of liquid, is added to the grinding mixture.

As mentioned above, part of the invention is the provision of a cocrystal of (R)-benzyl 5-methyl-1,4-diazepane-1-carboxylate hydrochloride with (R)-(+)-1,1,2-triphenyl-1,2-ethanediol ((R)-TED), of formula (II), which has a molar ratio of (R)-benzyl 5-methyl-1,4-diazepane-1-carboxylate hydrochloride: (R)-(+)-1,1,2-triphenyl-1,2-ethanediol of 1:1.

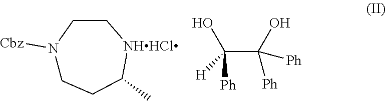

In formula (II), Cbz represents the benzyloxycarbonyl group.

In a preferred embodiment, the cocrystal of (R)-benzyl 5-methyl-1,4-diazepane-1-carboxylate hydrochloride with (R)-TED, of formula (II), is a crystalline form named Form A. This cocrystal Form A is easy to handle and shows crystal stability at room temperature.

The specific crystalline forms of the cocrystals of the present invention can be characterized by X-ray powder diffraction (XRPD), proton nuclear magnetic resonance analyses (¹H NMR), differential scanning calorimetry (DSC), and thermogravimetric analysis (TGA).

Diffraction measurements were performed at ambient conditions on a PANalyticalX'Pert PRO diffractometer with reflection θ-θ geometry, equipped with Cu K-alpha radiation and a PIXcel detector, operated at 45 kV and 40 mA. Each powder was mounted on a zero background silicon holder and allowed to spin during the data collection at 0.25 rev/s. The measurement angular range was 3.0-40.0° (2θ) with a step size of 0.013°. The scanning speed was 0.32826°/s.

Proton and carbon nuclear magnetic resonance analysis were recorded in deuterated chloroform in a Varian Mercury 400 spectrometer, equipped with a broadband probe ATB 1H/19F/X of 5 mm. Spectra were acquired dissolving 5-10 mg of sample in 0.6 mL of deuterated solvent.

DSC analysis was recorded with a Mettler DSC2. A sample of 2.4900 mg was weighed into a 40 μL aluminium crucible with a pinhole lid and was heated, under nitrogen (50 mL/min), at 10° C./min from 25 to 300° C.

Thermogravimetric analysis (TGA) was recorded in a Mettler TGA/SDTA851e thermogravimetric analyzer. A sample of 4.3400 mg was weighed into a 100 μL aluminium crucible and sealed with a lid. Samples were heated at 10° C./min from 25 to 550° C., under nitrogen (50 mL/min).

In a preferred embodiment, Form A of the cocrystal of (R)-benzyl 5-methyl-1,4-diazepane-1-carboxylate hydrochloride with (R)-TED, of formula (II), is characterized by having an X-ray diffractogram that comprises characteristic peaks at approximately 5.3, 10.6 and 15.8 degrees 2 theta at a Cu—K_{α} radiation, λ=1.5406 Å. In a more preferred embodiment, this cocrystal is characterized by further comprising characteristic peaks in the X-ray powder diffractogram at approximately 9.8, 12.9, and 19.7 degrees 2 theta at a Cu—K_{α} radiation, λ=1.5406 Å. In a still more preferred embodiment, this cocrystal is characterized by further comprising characteristic peaks in the X-ray powder diffractogram at approximately 6.5, 15.3, 15.8, 19.4, and 22.4 degrees 2 theta at a Cu—K_{α} radiation, λ=1.5406 Å.

More specifically, this new cocrystal Form A is characterized by exhibiting in the X-ray powder diffractogram a pattern of peaks, expressed in 2 theta units in degrees, 2θ (°), which is shown in Table 1.

TABLE 1
List of selected peaks (only peaks with relative
intensity greater than or equal to 1% are indicated):

| Pos. [°2Th.] | d-spacing [Å] | Rel. Int. [%] |
|---|---|---|
| 5.3 | 16.6 | 00 |
| 6.4 | 13.8 | 52 |
| 6.5 | 13.6 | 39 |
| 9.9 | 9.0 | 10 |
| 10.6 | 8.3 | 42 |
| 11.9 | 7.5 | 11 |
| 12.9 | 6.9 | 74 |
| 13.0 | 6.8 | 31 |
| 14.5 | 6.1 | 10 |
| 15.3 | 5.8 | 45 |
| 15.8 | 5.6 | 39 |
| 17.5 | 5.1 | 30 |
| 18.0 | 4.9 | 42 |
| 18.7 | 4.8 | 26 |
| 19.4 | 4.6 | 35 |
| 19.8 | 4.5 | 23 |
| 20.5 | 4.3 | 29 |
| 20.6 | 4.3 | 26 |
| 21.4 | 4.2 | 85 |
| 22.4 | 4.0 | 72 |
| 23.1 | 3.9 | 7 |
| 23.8 | 3.7 | 10 |
| 24.3 | 3.7 | 31 |
| 25.7 | 3.5 | 47 |
| 26.5 | 3.4 | 20 |
| 28.4 | 3.1 | 22 |
| 28.6 | 3.1 | 18 |
| 29.0 | 3.1 | 6 |
| 29.6 | 3.0 | 9 |
| 30.5 | 2.9 | 5 |
| 30.8 | 2.9 | 4 |
| 31.9 | 2.8 | 2 |
| 32.7 | 2.7 | 4 |
| 33.4 | 2.7 | 4 |
| 34.2 | 2.6 | 6 |
| 35.2 | 2.6 | 3 |
| 35.8 | 2.5 | 4 |
| 37.5 | 2.4 | 2 |
| 39.4 | 2.3 | 2 |

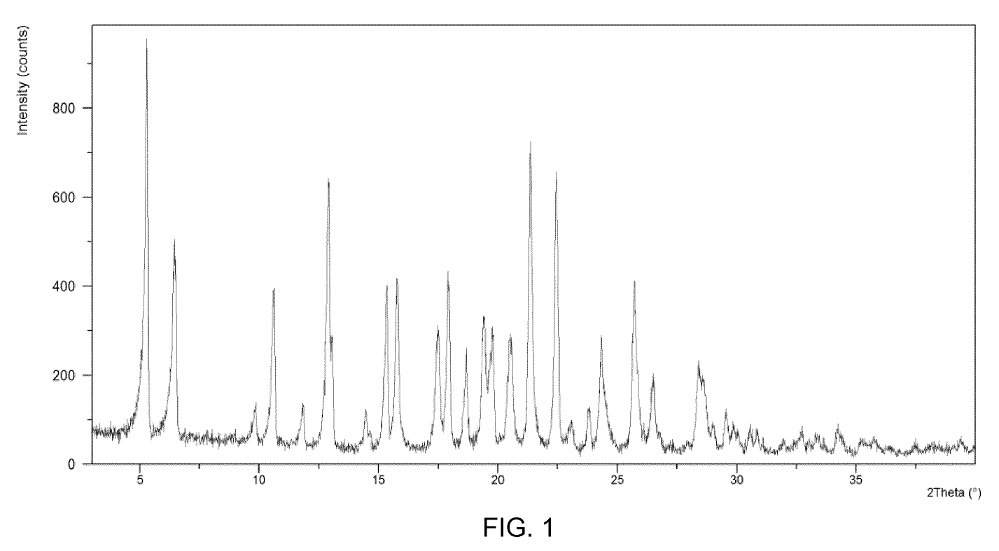

This cocrystal Form A may be further characterized by an X-ray diffractogram as in FIG. 1.

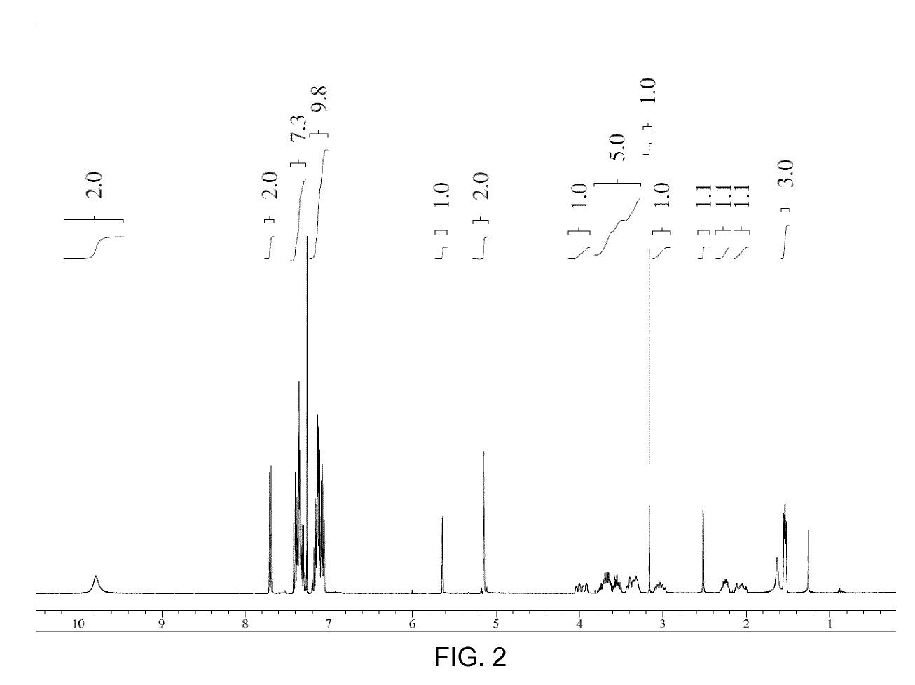

This cocrystal Form A may also be further characterized by the following ¹H NMR spectrum 1H NMR (CDCl₃, 400 MHz): δ=9.78 (s br, 2H), 7.73-7.67 (m, 2H arom.), 7.43-7.27 (m, 8H arom.), 7.21-7.03 (m, 10H arom.), 5.66-5.62 (m, 1H), 5.19-5.09 (m, 2H, Ph-CH₂—CO), 4.06-3.89 (m, 1H), 3.79-3.49 (m, 3H), 3.46-3.25 (m, 2H), 3.16 (s, 1H, −OH) 3.12-2.90 (m, 1H), 2.52 (d, 1H, —OH), 2.33-2.18 (m, 1H), 2.15-1.97 (m, 1H), 1.57-1.51 (m, 3H, —CH₃). This cocrystal Form A may be further characterized by a ¹H NMR spectrum as in FIG. 2.

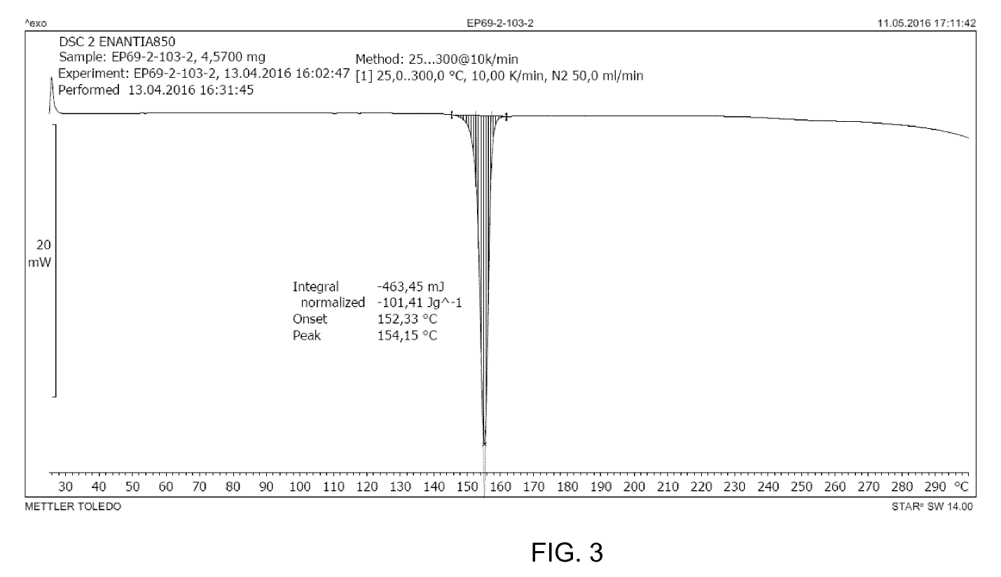

The cocrystal Form A may also be further characterized by an endothermic sharp peak corresponding to the melting point with an onset at about 152° C. (fusion enthalpy −107.89 J/g) measured by DSC analysis. This cocrystal Form A may be further characterized by DSC analysis as in FIG. 3.

The ratio (R)-benzyl 5-methyl-1,4-diazepane-1-carboxylate.HCl: (R)-TED in the cocrystal may be determined by ¹H NMR, titration, or elemental analysis.

A preliminary process which was used for the preparation of a cocrystal of (R)-benzyl 5-methyl-1,4-diazepane-1-carboxylate hydrochloride with (R)-TED, of formula (II), as defined above, comprises: wet grinding: either a) a mixture of a hydrochloride salt of (rac)-benzyl 5-methyl-1,4-diazepane-1-carboxylate of formula (rac)-(III) and (R)-TED; or alternatively, b) a mixture of (rac)-benzyl 5-methyl-1,4-diazepane-1-carboxylate, (R)-TED, and hydrochloric acid; in a solvent selected from the group consisting of acetonitrile, isopropanol, ethyl acetate, acetone, tetrahydrofuran, tert-butyl methyl ether; dichloromethane and toluene. In a particular embodiment, the solvent used is acetonitrile.

As mentioned above, part of the invention is a process for preparing a cocrystal of (R)-benzyl 5-methyl-1,4-diazepane-1-carboxylate hydrochloride with (R)-TED of formula (II) which comprises: a) combining either a₁ₐ) a mixture of a hydrochloride salt of (rac)-benzyl 5-methyl-1,4-diazepane-1-carboxylate of formula (rac)-(III) and (R)-TED, or alternatively, a_{1b}) (rac)-benzyl 5-methyl-1,4-diazepane-1-carboxylate of formula (rac)-(III), (R)-TED, and hydrochloric acid; in a solvent selected from the group consisting of acetonitrile, isopropanol, ethyl acetate, acetone, tetrahydrofuran, tert-butyl methyl ether and toluene; ₐ₂) either heating the mixture until complete dissolution or slurrying between room temperature and reflux; a₃) cooling down this mixture, if necessary; a₄) isolating the cocrystal of steps a₂) or a₃) thus obtained.

Since the hydrochloric acid may be aqueous hydrochloric acid, the solvent system may be a mixture of any of the solvents mentioned above (acetonitrile, isopropanol, ethyl acetate, acetone, tetrahydrofuran, tert-butyl methyl ether; and toluene) with water, preferably ACN and water. In particular, the amount of water in the mixture of solvent/water may be comprised up to 10% v/v. In a particular embodiment, the amount of water is comprised between 5-10% v/v.

In a particular embodiment, the solvent used is acetonitrile. In another particular embodiment, the cocrystal is obtained by slurrying in acetonitrile. In another particular embodiment, the cocrystal is obtained by crystallization from acetonitrile. Thus, a hot solution, preferably at reflux temperature is slowly cooled down to crystallize the cocrystal. The solution can be seeded to facilitate the crystallization.

(rac)-Benzyl 5-methyl-1,4-diazepane-1-carboxylate ((rac)-(III)) can be prepared using the methods described in Cox et al., J. Med. Chem. 2010, 53, 5320-5332 or in WO2008069997A1 (see scheme E of this patent application). The (rac)-(III) thus obtained can be transformed into its hydrochloride salt by known methods, for instance, by reacting it with hydrochloric acid in an appropriate solvent. (R)-TED is commercially available (CAS number 95061464) and can also be easily and economically obtained from mandelic acid which is a cheap raw material by methods known in the art.

The cocrystals of the present invention may be purified by recrystallization. Thus, after its preparation, a cocrystal can be submitted to a further recrystallization. In a preferred embodiment, the resolution process further comprises one recrystallization step. In a particular embodiment, the cocrystal of the present invention is Form A.

They can also be submitted to a slurrying process. In both cases (recrystallization or slurrying) the solvent is selected from the group consisting of acetonitrile, isopropanol, ethyl acetate, acetone and toluene. In a particular embodiment, the solvent used is acetonitrile. The same solvent can be used for the preparation step and for the recrystallization step or for the slurrying step.

In a preferred embodiment, Form A of the cocrystal of (R)-benzyl 5-methyl-1,4-diazepane-1-carboxylate hydrochloride with (R)-TED of formula (II) is prepared using acetonitrile as solvent.

The cocrystal of (R)-benzyl 5-methyl-1,4-diazepane-1-carboxylate hydrochloride with (R)-TED of the invention may also be defined by its preparation process. Accordingly, this aspect of the invention can be formulated as cocrystal of (R)-benzyl 5-methyl-1,4-diazepane-1-carboxylate hydrochloride with (R)-TED, as defined above, obtainable by the process disclosed above, optionally including any preferred or particular embodiments of the process, and possible combinations of some of the process features disclosed above.

The use of a cocrystal of (R)-benzyl 5-methyl-1,4-diazepane-1-carboxylate hydrochloride with (R)-TED, of formula (II), as an intermediate for the preparation of (R)-suvorexant or a pharmaceutically acceptable salt thereof is also part of the invention. Suvorexant may be obtained with ≥99% ee. The cocrystals of the present invention allow the resolution of a key intermediate in the preparation of suvorexant.

Accordingly, the resolution process of (rac)-benzyl 5-methyl-1,4-diazepane-1-carboxylate or a hydrochloride salt thereof is also part of the invention and comprises: a) preparing a cocrystal of (R)-benzyl 5-methyl-1,4-diazepane-1-carboxylate hydrochloride with (R)-TED), of formula (II), by the process disclosed above, optionally, either recrystallizing or slurrying the cocrystal thus obtained; and b) dissociating the cocrystal thus obtained to yield (R)-benzyl 5-methyl-1,4-diazepane-1-carboxylate hydrochloride salt, if desired, converting the salt into its free base of formula (R)-(III) and, if desired, converting the resulting free base into a salt thereof by reacting it with an appropriate acid.

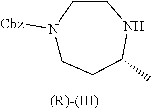 [SMILES: C[C@@H]1CCN(C(=O)OCc2ccccc2)CCN1]

In a preferred embodiment, the resolution process is carried out from racemic benzyl 5-methyl-1,4-diazepane-1-carboxylate free base as starting material. In another preferred embodiment, the resolution process is carried out from the hydrochloride salt of racemic benzyl 5-methyl-1,4-diazepane-1-carboxylate as starting material.

In a particular embodiment of the resolution process, the dissociation step comprises: (1) slurrying the cocrystal of (R)-benzyl 5-methyl-1,4-diazepane-1-carboxylate hydrochloride with (R)-TED, of formula (II), in water at room temperature; (2) separating the (R)-TED from the medium; and (3) basifying the aqueous phase, and extracting the (R)-benzyl 5-methyl-1,4-diazepane-1-carboxylate with an appropriate organic solvent. The solution thus obtained may be used; directly for the next step. Alternatively, the (R)-benzyl 5-methyl-1,4-diazepane-1-carboxylate free base can be isolated from the solution. As an example, the combined organic phases may be concentrated to dryness under vacuum at room temperature affording (R)-(III) free base as a colorless oil. If desired, a salt of (R)-benzyl 5-methyl-1,4-diazepane-1-carboxylate free base may be prepared by reacting the free base with an appropriate acid.

The aqueous phase which contains the (R)-benzyl 5-methyl-1,4-diazepane-1-carboxylate hydrochloride salt may be basified, for instance, with sodium hydroxide 1 M (pH 12), and may be extracted with, for instance, dichloromethane, preferably twice.

In another particular embodiment, the dissociation step comprises: (1) slurrying the cocrystal of (R)-benzyl 5-methyl-1,4-diazepane-1-carboxylate hydrochloride with (R)-TED, of formula (II), in water at room temperature; (2) separating the (R)-TED from the medium; and (3) concentrating the aqueous phase to isolate the (R)-benzyl 5-methyl-1,4-diazepane-1-carboxylate hydrochloride salt.

When the cocrystal of (R)-benzyl 5-methyl-1,4-diazepane-1-carboxylate.HCl with (R)-TED, Form A, is dissociated by slurrying in water at room temperature, the (R)-benzyl 5-methyl-1,4-diazepane-1-carboxylate hydrochloride remains dissolved in water and (R)-TED, insoluble in water, crystallizes. The crystalline (R)-TED may be recovered from the reaction medium, for instance, by filtration or by extraction in an organic solvent.

The enantiomeric excess of (R)-benzyl 5-methyl-1,4-diazepane-1-carboxylate free base can be determined as explained in the Examples section. The results show that the process of the present invention (see, for instance Examples 4, 5 and 8) can afford a higher global yield than the resolution method described in WO2016020404A1 (up to 30% instead of 20%) with an excellent enantiomeric excess (>99% ee instead of 96% ee).

The other enantiomer (S)-(III) or its hydrochloride salt can be resolved in the same way using the other enantiomer of TED, i.e. (S)-TED. It has the same X-ray, DSC and TGA as its enantiomer.

The chirality of each of the compounds can be determined as illustrated in Example 9.

The (R)-benzyl 5-methyl-1,4-diazepane-1-carboxylate (R)-(III) or its salts as defined above and obtained by the process of the invention, may be easily converted into suvorexant or its pharmaceutically acceptable salts by methods known in the art. Two different approaches can be followed for this conversion.

One approach involves a) N-acylation of (R)-benzyl 5-methyl-1,4-diazepane-1-carboxylate (R)-(III) or its salts with a benzoic acid derivative of formula (IV) wherein X is OH or Cl; b) N-deprotection to provide compound (VI); and c) coupling with a benzoxazole derivative compound (VII). This approach is applied, for instance, in WO20080699997 or in J. Med. Chem. 2010, 53, 5320-5332.

Accordingly, also part of the invention is a process for preparing suvorexant of formula (I) or a pharmaceutically acceptable salt thereof, which comprises: a) carrying out the resolution process as defined above, b) N-acylating the compound of formula (R)-(III) thus obtained with a benzoic acid derivative of formula (IV) wherein X is OH or CI, to yield a compound of formula (V); c) deprotecting the amino group of the compound of formula (V) thus obtained to provide a compound of formula (VI); d) coupling the compound obtained in step c) with a benzoxazole derivative of formula (VII) wherein Y is H, Cl or Br; to yield suvorexant (I) or a pharmaceutically acceptable salt thereof; and e) if desired, converting the resulting suvorexant free base into a pharmaceutically acceptable salt thereof by reacting it with an acceptable acid.

Step b) of the previous process may be carried out in the presence of a base, particularly, when the starting material is (R)-(III) hydrochloride salt. Examples of appropriate bases are: tertiary amines (eg. triethylamine or diisopropylethylamine) or inorganic bases (eg. alkali metal carbonates such as potassium carbonate).

When in compound of formula (IV) X is OH, the coupling can be effected with coupling agents well known to a person skilled in the art.

Example procedures for carrying out the coupling where Y is H, Cl or Br can be found in WO2015008218, WO2008069997 and WO2013169610, respectively.

This approach is illustrated in the following scheme:

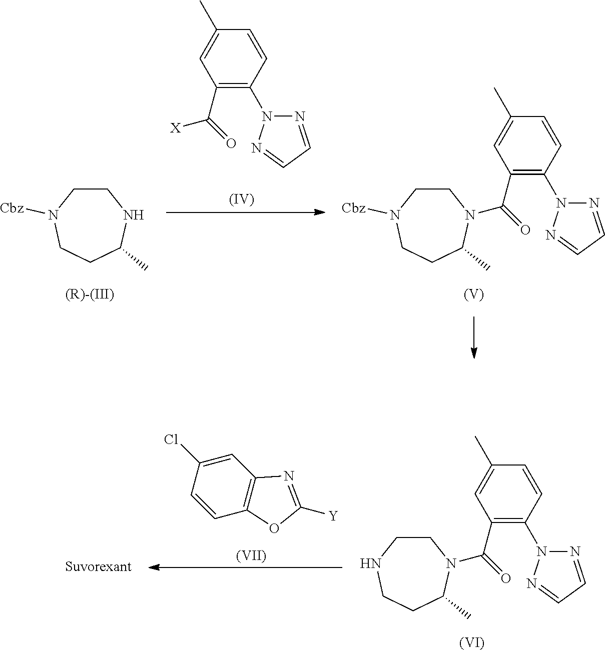

In the formula (R)-(III) and (V) Cbz means benzyloxycarbonyl.

Another approach encompasses the N-protection of the compound (R)-(III) as defined above with an orthogonal protecting group PG, followed by N-Cbz cleavage. Then, suvorexant can be obtained by a process involving a) coupling with a benzoxazole derivative of formula (VII); b) N-deprotection to provide compound (XI); and c) coupling with a benzoic acid derivative (IV). Aspects of this approach are applied, for instance, in WO2016020404A1, WO2015008218A2 and WO2012148553A1 where X and Y are defined as described above.

Accordingly, also part of the invention is a process for preparing suvorexant of formula (I) or a pharmaceutically acceptable salt thereof, which comprises: a) carrying out the resolution process disclosed above, b) N-protecting a compound of formula (R)-(III) thus obtained with an orthogonal protecting group PG (i.e. a protecting group which can be removed selectively in the presence of Cbz) to yield a compound of formula (VIII); c) cleavage of Cbz protecting group to yield a compound of formula (IX); d) coupling the compound thus obtained with a benzoxazole derivative of formula (VII) wherein Y is H, CI or Br to yield a compound of formula (X); e) N-deprotecting the compound of formula (X) to yield a compound of formula (XI); f) coupling the compound of formula (XI) with a benzoic acid derivative of formula (IV) where X is OH or Cl (coupling where X=OH can be effected with coupling agents well known to a person skilled in the art), to yield suvorexant or a pharmaceutically acceptable salt thereof; and, if desired, g) converting the resulting suvorexant free base into a pharmaceutically acceptable salt thereof by reacting it with a pharmaceutically acceptable acid.

Step b) of the previous process may be carried out in the presence of a base, particularly, when the starting material is (R)-(III) hydrochloride salt. Examples of appropriate bases are: tertiary amines (eg. triethylamine or diisopropylethylamine) or inorganic bases (eg. alkali metal carbonates such as potassium carbonate).

This approach is illustrated in the following scheme:

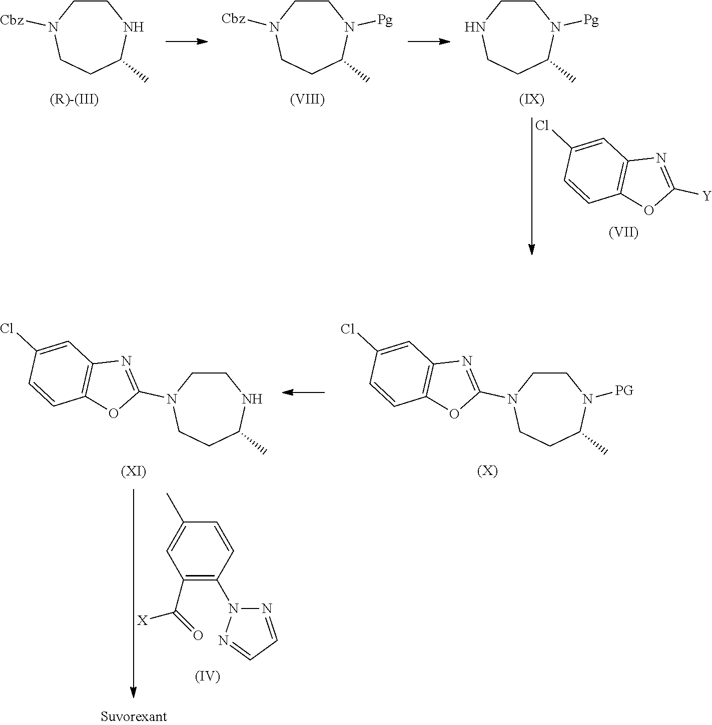

In a particular embodiment of the processes for the preparation of suvorexant disclosed above X in compound (IV) is chlorine. In another particular embodiment of the processes for the preparation of suvorexant, Y in compound (VII) is bromine or chlorine.

The deprotection of the Cbz group can be carried out, for instance by hydrogenolysis with H₂, Pd(OH)₂, EtOAc. This amino protective group can be introduced and removed by other procedures known in the art (cf. T. W. Greene and G. M. Wuts, Protective Groups in Organic Synthesis, Third Edition, Wiley, N.Y., 1999, see pages 531-535).

The group PG is a suitable orthogonal amino protecting group. The term “suitable orthogonal amino protecting group” as used herein is denoted to encompass any amino protecting group other than the protective group Cbz used to protect the other amino group of compound of formula (R)-(III), stable to selected Cbz cleavage conditions (for example, hydrogenolysis). Representative protecting groups for amino groups are well known to those skilled in the art and are described, for example, in T. W. Greene and G. M. Wuts, Protective Groups in Organic Synthesis, Third Edition, Wiley, N.Y., 1999 (see chapter 7). Preferred protecting groups for PG include, but are not limited to, carbamate-forming groups such as Boc (tert-butyloxycarbonyl), Fmoc (9-fluorenylmethyloxycarbonyl), methyl and ethyl carbamates; sulfonamide-forming groups such as tosyl; and amide-forming groups such as formyl, (un)substituted acetyl, and benzoyl. In a preferred embodiment, PG is Boc.

The coupling of compound (IX) with compound (VII, Y=Cl or Br) and the coupling of compound of formula (XI) with a benzoic acid derivative of formula (IV, X=Cl) can be carried out, for instance, in the presence of a base, such as triethylamine, and an appropriate solvent such as dichloromethane.

The coupling of compound (IX) with compound (VII, Y=H) can be carried out, for instance, in the presence of Cu(OAc)₂ in acetic acid and acetonitrile according to WO2015008218A2.

The coupling of compound (XI) with a benzoic acid derivative of formula (IV, X=OH) can be carried out in the presence of standard amide-forming coupling reagents known to a person expert in the art.

The term “pharmaceutically acceptable salts” refers to salts prepared by methods known in the art from pharmaceutically acceptable non-toxic acids including inorganic or organic acids. Such acids include acetic, benzene sulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, or p-toluenesulfonic acid.

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples and drawings are provided by way of illustration, and they are not intended to be limiting of the present invention. Furthermore, the present invention covers all possible combinations of particular and preferred embodiments described herein.

### EXAMPLES

### Example 1: Detection of the Cocrystal of (R)-benzyl 5-methyl-1,4-diazepane-1-carboxylate hydrochloride with (R)-(+)-1,1,2-triphenyl-1,2-ethanediol Form A and of the Cocrystal of (S)-benzyl 5-methyl-1,4-diazepane-1-carboxylate hydrochloride with (S)-(−)-1,1,2-triphenyl-1,2-ethanediol

The starting materials used are (rac)-benzyl 5-methyl-1,4-diazepane-1-carboxylate.HCl and (R)-TED (1:1) or (S)-TED (1:1).

A cocrystal of (S)-benzyl 5-methyl-1,4-diazepane-1-carboxylate hydrochloride with (S)-(−)-1,1,2-triphenyl-1,2-ethanediol was obtained by both slurrying and by wet grinding in acetonitrile (ACN).

A cocrystal of (S)-benzyl 5-methyl-1,4-diazepane-1-carboxylate hydrochloride with (S)-(−)-1,1,2-triphenyl-1,2-ethanediol was also obtained by slurrying with isopropanol, ethyl acetate, acetone and toluene as solvent and also by wet grinding with isopropanol, ethyl acetate, acetone, tetrahydrofuran, tert-butyl methyl ether, dichloromethane and toluene.

The cocrystal of (R)-benzyl 5-methyl-1,4-diazepane-1-carboxylate hydrochloride with (R)-(+)-1,1,2-triphenyl-1,2-ethanediol Form A may be obtained by slurrying and by grinding in ACN (rac)-benzyl 5-methyl-1,4-diazepane-1-carboxylate.HCl with (R)-TED (1:1).

The cocrystal of (R)-benzyl 5-methyl-1,4-diazepane-1-carboxylate hydrochloride with (R)-(+)-1,1,2-triphenyl-1,2-ethanediol Form A may also be obtained by slurrying with isopropanol, ethyl acetate, acetone, and toluene as solvent and also by wet grinding with isopropanol, ethyl acetate, acetone, tetrahydrofuran, tert-butyl methyl ether, dichloromethane, and toluene.

### Example 2: Preparation of a Cocrystal of (S)-benzyl 5-methyl-1,4-diazepane-1-carboxylate hydrochloride with (S)-(−)-1,1,2-triphenyl-1,2-ethanediol and of the Cocrystal of (R)-benzyl 5-methyl-1,4-diazepane-1-carboxylate hydrochloride with (R)-(+)-1,1,2-triphenyl-1,2-ethanediol Form A by slurrying in acetonitrile (ACN)

In an Eppendorf, racemic benzyl 5-methyl-1,4-diazepane-1-carboxylate hydrochloride (19.8 mg, 0.07 mmols) and (S)-1,1,2-triphenyl-1,2-ethandiol (20.1 mg, 0.07 mmols) were suspended in acetonitrile (0.2 mL). The resulting suspension was left stirring at room temperature for 15 hours (overnight). Then, the solid was recovered by centrifuge and dried under high vacuum at room temperature.

(R)-Benzyl 5-methyl-1,4-diazepane-1-carboxylate hydrochloride with (R)-(+)-1,1,2-triphenyl-1,2-ethanediol Form A may be obtained by the same process starting from (R)-1,1,2-triphenyl-1,2-ethandiol instead of (S)-1,1,2-triphenyl-1,2-ethandiol.

### Example 3: Preparation of a Cocrystal of (S)-benzyl 5-methyl-1,4-diazepane-1-carboxylate hydrochloride with (S)-(−)-1,1,2-triphenyl-1,2-ethanediol and of the Cocrystal of (R)-benzyl 5-methyl-1,4-diazepane-1-carboxylate hydrochloride with (R)-(+)-1,1,2-triphenyl-1,2-ethanediol Form A by wet grinding in ACN

In an Eppendorf, (rac)-benzyl 5-methyl-1,4-diazepane-1-carboxylate hydrochloride (20.3 mg, 0.07 mmols), (S)-1,1,2-triphenyl-1,2-ethandiol (20.2 mg, 0.07 mmols), two drops of acetonitrile and three steel balls were combined. The resulting mixture was milled three times for 10 minutes at 30 MHz in a grinding mortar and the solid was dried under high vacuum at room temperature.

(R)-Benzyl 5-methyl-1,4-diazepane-1-carboxylate hydrochloride with (R)-(+)-1,1,2-triphenyl-1,2-ethanediol Form A may be obtained by the same process starting from (R)-1,1,2-triphenyl-1,2-ethandiol instead of (S)-1,1,2-triphenyl-1,2-ethandiol.

### Example 4: Preparation of the Cocrystal of (R)-benzyl 5-methyl-1,4-diazepane-1-carboxylate hydrochloride with (R)-(+)-1,1,2-triphenyl-1,2-ethanediol, of Formula (II), Form A, by Crystallization from ACN

In a round-bottomed 10 mL flask, (rac)-benzyl 5-methyl-1,4-diazepane-1-carboxylate hydrochloride (1 g, 3.52 mmol) and (R)-1,1,2-triphenyl-1,2-ethandiol (1.027 g, 3.53 mmol, 1.0 equiv.) were suspended in acetonitrile (6.0 mL). The resulting mixture was heated to reflux. The resulting clear solution was stirred for 10 min at reflux before cooling slowly to room temperature. During the cooling, the mixture was seeded by Form A each 10° C. and a white solid started to crystallize at 60° C. Once at room temperature, the resulting suspension was stirred 3 h at room temperature and 1 h at 0-5° C. with an ice bath before isolating the solid. The white solid was filtered in a sintered funnel (no. 3), washed twice with cold acetonitrile (2×1.0 mL) and dried under high vacuum at room temperature to provide Form A (835 mg, 41% yield, 88% ee) as a white solid.

### Example 5: Recrystallization of the Cocrystal of (R)-benzyl 5-methyl-1,4-diazepane-1-carboxylate hydrochloride with (R)-(+)-1,1,2-triphenyl-1,2-ethanediol Form A from ACN

In a round-bottomed 10 mL flask equipped with magnetic stirring, the cocrystal of (R)-benzyl 5-methyl-1,4-diazepane-1-carboxylate hydrochloride with (R)-(+)-1,1,2-triphenyl-1,2-ethanediol Form A (801.4 mg, 1.39 mmol, 88% ee) was suspended in acetonitrile (4.8 mL). The resulting mixture was heated to reflux. The resulting clear solution was stirred for 10 min at reflux before cooling slowly to room temperature. During the cooling, the mixture was seeded by Form A each 10° C. and a white solid started to crystallize at 60° C. Once at room temperature, the resulting suspension was stirred 3 h at room temperature and 1 h at 0-5° C. with an ice bath before isolating the solid. The white solid was filtered in a sintered funnel (no. 3), washed twice with cold acetonitrile (2×0.8 mL) and dried under high vacuum at room temperature to provide Form A (639 mg, 80% yield, >99% ee) as a white solid.

Seeding crystals may be obtained by any of the processes disclosed above. They can additionally be obtained from the wet grinding process disclosed above but using (R)-benzyl 5-methyl-1,4-diazepane-1-carboxylate of formula (R)-(III) as starting material.

### Example 6: Crystallization of the Cocrystal of (R)-benzyl 5-methyl-1,4-diazepane-1-carboxylate hydrochloride with (R)-(+)-1,1,2-triphenyl-1,2-ethanediol Form A and of the Cocrystal of (S)-benzyl 5-methyl-1,4-diazepane-1-carboxylate hydrochloride with (S)-(−)-1,1,2-triphenyl-1,2-ethanediol from ACN

Several assays were carried out crystallizing the cocrystals in 7 vol. ACN in the conditions shown in Table 1.

TABLE 1

| Exp. | TED | Eq.-TED | Scale | Yield | % ee |
|---|---|---|---|---|---|
| 6A | S | 1 | 100 mg | 33% | 84 |
| 6B | S | 2 | 100 mg | 43% | 79 |
| 6C | S | 1 | 500 mg | 41% | 75 |
| 6D | R | 1 | 500 mg | 39% | 81 |
| 6E | R | 1 | 1 g | 41% | 87 |

### Example 7: Recrystallization of the Cocrystal of (R)-benzyl 5-methyl-1,4-diazepane-1-carboxylate hydrochloride with (R)-(+)-1,1,2-triphenyl-1,2-ethanediol Form A and of the Cocrystal of (S)-benzyl 5-methyl-1,4-diazepane-1-carboxylate hydrochloride with (S)-(−)-1,1,2-triphenyl-1,2-ethanediol from ACN

Several assays were carried out recrystallizing the cocrystal in 6 vol. ACN in the conditions shown in Table 2.

TABLE 2

| Exp. | From | Scale | Yield | % ee |
|---|---|---|---|---|
| 7A | 6A (84% ee) | 50 mg | 52% | >99 |
| 7B | 6B (79% ee) | 50 mg | 52% | >99 |
| 7C | 6C (75% ee) | 400 mg | 59% | >99 |
| 7D | 6D (81% ee) | 350 mg | 74% | >99 |
| 7E | 6E (87% ee) | 800 mg | 80% | >99 |

### Example 8: Preparation of (R)-benzyl 5-methyl-1,4-diazepane-1-carboxylate Free Base by Dissociation of the Cocrystal of (R)-benzyl 5-methyl-1,4-diazepane-1-carboxylate hydrochloride with (R)-(+)-1,1,2-triphenyl-1,2-ethanediol Form A

In a round-bottomed 10 mL flask equipped with magnetic stirring, Form A (94 mg, 0.164 mmols, >99% ee) was suspended in cold water (2.0 mL) and stirred for 1 h at 0-4° C. in an ice bath. (R)-TED was filtered using a sintered funnel (no. 3), washed twice with cold water (2×0.5 mL) and dried under high vacuum at room temperature affording (R)-1,1,2-triphenyl-1,2-ethandiol (44 mg, 92% recovery) as a white crystalline solid.

The combined aqueous mother liquor was washed with TBME (2×1 mL) before adding dichloromethane (3 mL) and a solution of 1 M sodium carbonate until pH 10. After a vigorous stirring, the phases were separated and the aqueous phase was extracted twice with dichloromethane (2×3 mL). The combined organic phases were washed with water (3 mL) and brine (3 mL), and dried over anhydrous sodium sulphate. The solvent was distilled at room temperature under vacuum to obtain (R)-benzyl 5-methyl-1,4-diazepane-1-carboxylate free base (40 mg, 84% yield, 99% ee) as a pale yellow oil.

### Example 9: Determination of the Chiral Configuration of (R)-benzyl 5-methyl-1,4-diazepane-1-carboxylate Free Base

The chiral configuration of (R)-benzyl 5-methyl-1,4-diazepane-1-carboxylate free base was determined by:

a) Reproducing the resolution of (rac)-benzyl 5-methyl-1,4-diazepane-1-carboxylate with DBTA described in the Sandoz patent (WO2016020404A1, see for instance from page 17 onwards and from page 70 onwards), allowed comparison of (R)-III retention time by chiral HPLC analysis with that of Form A, and confirmed that (R)-TED forms a cocrystal with (R)-III.HCl.

Analytical HPLC analyses were carried out on a Chiralpak IA column (5 μm, 4.6 mm×250 mm) with a mixture of heptane/EtOH-0.2% DEA 90:10 as eluent and at a flow rate of 1 mL/min ((R)-III is the first eluting enantiomer—Tr (R)-III 12.0 min and (S)-III) 14.0 min).

b) Protecting the second amine of (R)-III free base with a Boc group, measuring the specific rotation ([α]_{D}−24.0 (c 1.10, chloroform)), and comparing it to literature values ([α]_{D}−24.3 (c 1.0, chloroform), see. Cox et al., J. Med. Chem. 2010, vol. 53, pp. 5320-5332).

### CITATION LIST

### Patent Literature

WO2008069997A1
WO2016020404A1
WO2015008218A2
WO2012148553A1

### Non Patent Literature

J. Med. Chem. 2010, vol. 53, pp. 5320-5332

1. A cocrystal of (R)-benzyl 5-methyl-1,4-diazepane-1-carboxylate hydrochloride with (R)-(+)-1,1,2-triphenyl-1,2-ethanediol ((R)-TED) of formula (II) wherein Cbz is benzyloxycarbonyl, which has a molar ratio of (R)-benzyl 5-methyl-1,4-diazepane-1-carboxylate hydrochloride: (R)-(+)-1,1,2-triphenyl-1,2-ethanediol of 1:1.

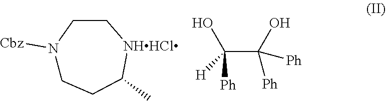

2. The cocrystal according to claim 1, which is a crystalline form named Form A, and is characterized by having an X-ray diffractogram that comprises characteristic peaks at approximately 5.3, 10.6, and 15.8 degrees 2 theta at a Cu—K_{α} radiation, λ=1.5406 Å.

3. The cocrystal according to claim 2, which is characterized by further comprising characteristic peaks in the X-ray powder diffractogram at approximately 9.8, 12.9, and 19.7 degrees 2 theta at a Cu—K_{α} radiation, λ=1.5406 Å.

4. The cocrystal according to claim 3, characterized in that the endothermic sharp peak corresponding to the melting point has an onset at about 152° C.

5. A resolution process of (rac)-benzyl 5-methyl-1,4-diazepane-1-carboxylate of formula (rac)-(III) or a salt thereof, which comprises
a) preparing a cocrystal of (R)-benzyl 5-methyl-1,4-diazepane-1-carboxylate hydrochloride with (R)-TED of formula (II) as defined in claim 1 by a process which comprises the steps of:
a₁) combining either a₁ₐ) a mixture of a hydrochloride salt of (rac)-benzyl 5-methyl-1,4-diazepane-1-carboxylate of formula (rac)-(III) and (R)-(+)-1,1,2-triphenyl-1,2-ethanediol ((R)-TED) or, alternatively, a_{1b}) (rac)-benzyl 5-methyl-1,4-diazepane-1-carboxylate of formula (rac)-(III), (R)-TED and hydrochloric acid; in a solvent selected from the group consisting of acetonitrile, isopropanol, ethyl acetate, acetone, tetrahydrofuran, tert-butyl methyl ether and toluene; 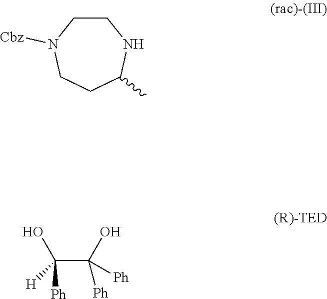 [SMILES: CC1CCN(C(=O)OCc2ccccc2)CCN1 ; O[C@H](c1ccccc1)C(O)(c1ccccc1)c1ccccc1]
a₂) either heating the mixture thus obtained until complete dissolution or, alternatively, slurrying between room temperature and reflux;
a₃) cooling down this mixture if necessary; and
a₄) isolating the cocrystal of formula (II) obtained in steps a₂) or a₃); and
b) converting the cocrystal obtained in step a₄) into (R)-benzyl 5-methyl-1,4-diazepane-1-carboxylate or a salt thereof.

6. The resolution process according to claim 5, further comprising a recrystallization step of the cocrystal obtained in step a₄) of claim 5, in a solvent selected from the group consisting of acetonitrile, isopropanol, ethyl acetate, acetone, and toluene.

7. The resolution process according to claim 5, wherein the solvent of the preparation step is acetonitrile.

8. The resolution process according to claim 5, wherein the solvent of the recrystallization step is acetonitrile.

9. The resolution process according to claim 5, wherein step b) comprises dissociating the cocrystal to yield (R)-benzyl 5-methyl-1,4-diazepane-1-carboxylate hydrochloride salt, if desired converting the salt into its free base of formula (R)-(III) and, if desired, converting the compound thus obtained to a salt thereof by reacting it with an appropriate acid

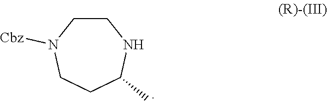 [SMILES: C[C@@H]1CCN(C(=O)OCc2ccccc2)CCN1]

10. The resolution process according to claim 9, wherein the dissociation step comprises:
(1) slurrying the cocrystal of (R)-benzyl 5-methyl-1,4-diazepane-1-carboxylate hydrochloride with (R)-TED of formula (II), in water between 0° C. and room temperature;
(2) separating the (R)-TED from the medium; and
(3) basifying the aqueous phase, extracting the (R)-benzyl 5-methyl-1,4-diazepane-1-carboxylate of formula (R)-(III) with an appropriate organic solvent; optionally isolating the (R)-benzyl 5-methyl-1,4-diazepane-1-carboxylate of formula (R)-(III) as a free base, and if desired converting the free base into a salt thereof by addition of the corresponding acid.

11. The resolution process according to claim 9, wherein the dissociation step comprises:
(1) slurrying the cocrystal of (R)-benzyl 5-methyl-1,4-diazepane-1-carboxylate hydrochloride with (R)-TED of formula (II), in water between 0° C. and room temperature;
(2) separating the (R)-TED from the medium; and
(3) isolating the (R)-benzyl 5-methyl-1,4-diazepane-1-carboxylate hydrochloride of formula (R)-(III).HCl from the aqueous phase.

12. A process for the preparation of (R)-suvorexant or a pharmaceutically acceptable salt thereof which comprises the use of the cocrystal of (R)-benzyl 5-methyl-1,4-diazepane-1-carboxylate hydrochloride with (R)-TED of formula (II), as defined in claim 1, as an intermediate of the process.

13. A process for preparing suvorexant of formula (I) or a pharmaceutically acceptable salt thereof, 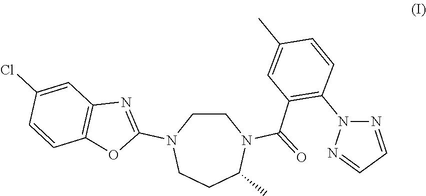 [SMILES: Cc1ccc(-n2nccn2)c(C(=O)N2CCN(c3nc4cc(Cl)ccc4o3)CC[C@H]2C)c1]
which comprises:
a) carrying out the resolution process of claim 5 to yield (R)-benzyl 5-methyl-1,4-diazepane-1-carboxylate of formula (R)-(III) or a hydrochloride salt thereof,
b) N-acylating a compound of formula (R)-(III), with a benzoic acid derivative of formula (IV) wherein X is OH or Cl, optionally in the presence of a base, 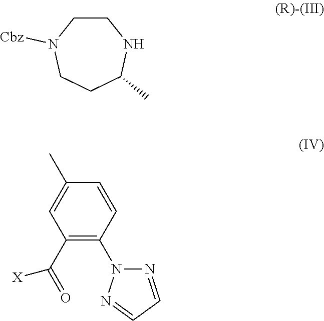
to yield a compound of formula (V); 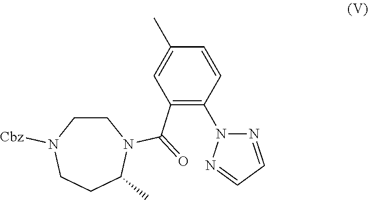 [SMILES: Cc1ccc(-n2nccn2)c(C(=O)N2CCN(C(=O)OCc3ccccc3)CC[C@H]2C)c1]
wherein Cbz in compound (R)-(III) and (V) is benzyloxycarbonyl;
c) deprotecting the amino group of the compound of formula (V) thus obtained to provide the compound of formula (VI); 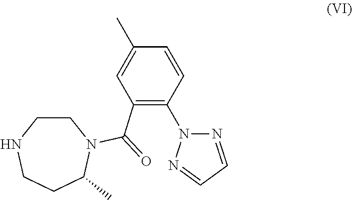 [SMILES: Cc1ccc(-n2nccn2)c(C(=O)N2CCNCC[C@H]2C)c1]
d) coupling the compound obtained in step c) with a benzoxazole derivative of formula (VII) wherein Y is H, Cl or Br; 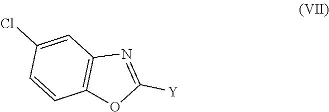
to yield suvorexant (I) or a pharmaceutically acceptable salt thereof; and
e) if desired, converting the resulting suvorexant free base into a pharmaceutically acceptable salt thereof by reacting it with a pharmaceutically acceptable acid.

14. A process for preparing suvorexant of formula (I) or a pharmaceutically acceptable salt thereof, 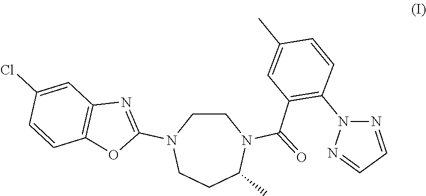 [SMILES: Cc1ccc(-n2nccn2)c(C(=O)N2CCN(c3nc4cc(Cl)ccc4o3)CC[C@H]2C)c1]
which comprises:
a) carrying out the resolution process of claim 5, to yield (R)-benzyl 5-methyl-1,4-diazepane-1-carboxylate of formula (R)-(III) or a hydrochloride salt thereof,
b) N-protecting a compound of formula (R)-(III) or its hydrochloride salt, optionally in the presence of a base, 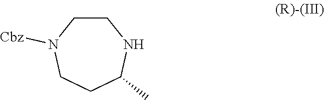 [SMILES: C[C@@H]1CCN(C(=O)OCc2ccccc2)CCN1]
with an orthogonal protecting group PG, to yield a compound of formula (VIII) 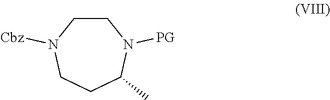
c) cleavage of the Cbz group to yield a compound of formula (IX); 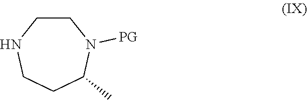
wherein PG is an amino protecting group;
d) coupling the compound thus obtained with a benzoxazole derivative of formula (VII), wherein Y is H, Cl or Br, 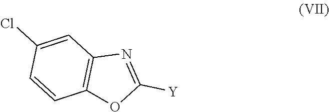
to yield a compound of formula (X) wherein PG is an amino protecting group; 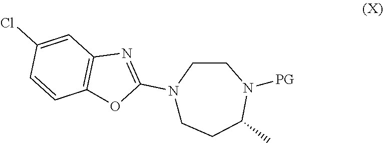
e) N-deprotecting the compound of formula (X) to yield a compound of formula (XI); 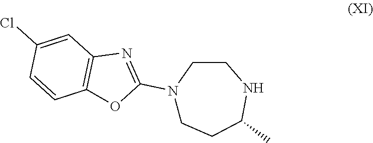 [SMILES: C[C@@H]1CCN(c2nc3cc(Cl)ccc3o2)CCN1]
f) coupling the compound of formula (XI) with a benzoic acid derivative of formula (IV) wherein X is OH or Cl, 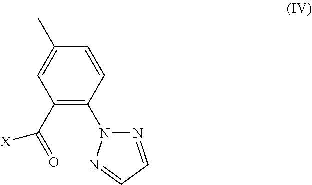
to yield suvorexant or a pharmaceutically acceptable salt thereof and; if desired,
g) converting the resulting suvorexant free base into a salt thereof by reacting it with a pharmaceutically acceptable acid.

15. The process according to claim 14, wherein PG is a tert-butoxycarbonyl protecting group.
